(19) United States
(12) Patent Application Publication
(10) Pub. No.: US 2017/0334913 A1
(43) Pub. Date: Nov. 23, 2017

(54) Methods Of Preparing Carbocyclic Nucleosides

(71) Applicant: Cellceutix Corporation, Beverly, MA (US)

(72) Inventors: Siya Ram, Beverly, MA (US); Krishna Menon, Beverly, MA (US)

(21) Appl. No.: 15/597,872

(22) Filed: May 17, 2017

Related U.S. Application Data
(60) Provisional application No. 62/338,006, filed on May 18, 2016.

(51) Int. Cl. C07D 473/16

(52) U.S. Cl. CPC C07D 473/16

(57) ABSTRACT

The present disclosure provides methods of preparing carbocyclic nucleosides, in particular, Prurisol™ ((−) cis-[4-[2-amino-6-(cyclopropylamino)-9H-purin-9-yl]-2-cyclopentene-1-hydroxymethyl acetate).

### CROSS-REFERENCE TO RELATED APPLICATIONS

This application claims priority to U.S. provisional application Ser. No. 62/338,006 filed May 18, 2016, which is incorporated herein by reference in its entirety.

### FIELD

The present disclosure is directed, in part, to methods of preparing carbocyclic nucleosides, in particular, Prurisol™ ((−) cis-[4-[2-amino-6-(cyclopropylamino)-9H-purin-9-yl]-2-cyclopentene-1-hydroxymethyl acetate).

### BACKGROUND

Abacavir, (−) cis-[4-[2-amino-6-cyclopropylamino)-9H-purin-9-yl]-2-cyclopenten-yl]-1-methanol, a carbocyclic nucleoside which possesses a 2,3-dehydrocyclopentene ring, is referred to in U.S. Pat. No. 5,034,394 as a reverse transcriptase inhibitor. Recently, a general synthetic strategy for the preparation of this type of compound and intermediates was reported (see, Crimmins, et. al., J. Org. Chem., 1996, 61, 4192-4193; and Crimmins, et. al., J. Org. Chem., 2000, 65, 8499-85090). PCT International Publication No. WO 2013/103601 reports esters of abacavir, including (−) cis-[4-[2-amino-6-(cyclopropylamino)-9H-purin-9-yl]-2-cyclopentene-1-hydroxymethyl acetate (also referred to as Prurisol™) and pharmaceutically acceptable salts thereof. Prurisol™ ((−) cis-[4-[2-amino-6-(cyclopropylamino)-9H-purin-9-yl]-2-cyclopentene-1-hydroxymethyl acetate) is an orally bioavailable compound for the treatment of inflammatory skin diseases such as psoriasis, eczema and soborrhiasis.

### SUMMARY

The present disclosure provides methods of preparing Prurisol™ ((−) cis-[4-[2-amino-6-(cyclopropylamino)-9H-purin-9-yl]-2-cyclopentene-1-hydroxymethyl acetate) comprising: a) adding 1-ethyl-3-(3-dimethylaminopropyl)carbodiimide (EDC-HCl) to a suspension comprising Abacavir free base, a coupling agent, and 4-dimethylaminopyridine (DMAP) in a solvent under conditions sufficient to produce ((1S, 4R)-4-(2-amino-6-(cyclopropylamino)-9H-purin-9-yl)cyclopent-2-en-1-yl)methyl 2-(tert-butoxy) acetate; and b) adding a strong acid to a solution of ((1S, 4R)-4-(2-amino-6-(cyclopropylamino)-9H-purin-9-yl) cyclopent-2-en-1-yl)methyl 2-(tert-butoxy) acetate in a solvent under conditions sufficient to produce Prurisol™ ((−) cis-[4-[2-amino-6-(cyclopropylamino)-9H-purin-9-yl]-2-cyclopentene-1-hydroxymethyl acetate). In some embodiments, the methods further comprise dissolving Prurisol™ ((−) cis-[4-[2-amino-6-(cyclopropylamino)-9H-purin-9-yl]-2-cyclopentene-1-hydroxymethyl acetate) in a mixture of acetone and water under conditions sufficient to produce crystallized Prurisol™ ((−) cis-[4-[2-amino-6-(cyclopropylamino)-9H-purin-9-yl]-2-cyclopentene-1-hydroxymethyl acetate). In some embodiments, the methods further comprise preparing 2-(tert-butoxy) acetic acid (CTOX) by adding a haloacetic acid to a suspension of t-butanol and potassium tert-butoxide.

### BRIEF DESCRIPTION OF THE DRAWINGS

FIG. 1 shows a representative schematic for a method of preparing Prurisol™ ((−) cis-[4-[2-amino-6-(cyclopropylamino)-9H-purin-9-yl]-2-cyclopentene-1-hydroxymethyl acetate).

### DESCRIPTION OF EMBODIMENTS

Unless defined otherwise, all technical and scientific terms have the same meaning as is commonly understood by one of ordinary skill in the art to which the embodiments disclosed belongs.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, un-recited elements or method steps. In some embodiments, the compositions, methods, and devices can also “consist essentially of” or “consist of” the various components and steps, and such terminology should be interpreted as defining essentially closed-member groups.

As used herein, the terms “a” or “an” means “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

As used herein, the term “halo” refers to halogen groups including, but not limited to fluoro, chloro, bromo, and iodo.

As used herein, the phrase “reaction chamber” refers to a vessel, reactor, or flask that is routinely used for chemical reactions, and refers to herein any vessel, reactor, or flask that can be used to carry out any one or more of the chemical reactions described herein. A suitable reaction chamber is, for example, a round bottom flask.

At various places in the present specification, various parameters may be disclosed in groups or in ranges. It is specifically intended that the particular parameter include each and every individual subcombination of the members of such groups and ranges. For example, the phrase “30° C. to 35° C.” is specifically intended to individually disclose 30° C., 31° C., 32° C., 33° C., 34° C., and 35° C., as well as the ranges 30° C. to 35° C., 30° C. to 34° C., 30° C. to 33° C., 30° C. to 32° C., 30° C. to 31° C., 31° C. to 35° C., 32° C. to 35° C., 33° C. to 35° C., 34° C. to 35° C., 32° C. to 34° C., 32° C. to 33° C., and 33° C. to 34° C.

It is further appreciated that certain features of the present disclosure, which are, for clarity, described in the context of separate embodiments, can also be provided in combination in a single embodiment. Conversely, various features of the present disclosure which are, for brevity, described in the context of a single embodiment, can also be provided separately or in any suitable subcombination.

The compounds described herein can be asymmetric (e.g., having one or more stereocenters). All stereoisomers, such as enantiomers and diastereomers, are intended to be included within the scope of the present disclosure unless otherwise indicated. Compounds of the present disclosure that contain asymmetrically substituted carbon atoms can be isolated in optically active or racemic forms. Methods of preparation of optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are also included within the scope of the present disclosure and can be isolated as a mixture of isomers or as separated isomeric forms. Where a compound capable of stereoisomerism or geometric isomerism is designated in its structure or name without reference to specific R/S or cis/trans configurations, it is intended that all such isomers are contemplated.

Resolution of racemic mixtures of compounds can be carried out by any of numerous methods known in the art, including, for example, fractional recrystallization using a chiral resolving acid which is an optically active, salt-forming organic acid. Suitable resolving agents for fractional recrystallization methods include, but are not limited to, optically active acids, such as the D and L forms of tartaric acid, diacetyltartaric acid, dibenzoyltartaric acid, mandelic acid, malic acid, lactic acid, and the various optically active camphorsulfonic acids such as β-camphorsulfonic acid. Other resolving agents suitable for fractional crystallization methods include, but are not limited to, stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like. Resolution of racemic mixtures can also be carried out by elution on a column packed with an optically active resolving agent (e.g., dinitrobenzoylphenylglycine). Suitable elution solvent compositions can be determined by one skilled in the art.

It is understood that the present disclosure encompasses the use, where applicable, of stereoisomers, diastereomers and optical stereoisomers of the compounds of the present disclosure, as well as mixtures thereof. Additionally, it is understood that stereoisomers, diastereomers, and optical stereoisomers of the compounds of the present disclosure, and mixtures thereof, are within the scope of the present disclosure. By way of non-limiting example, the mixture may be a racemate or the mixture may comprise unequal proportions of one particular stereoisomer over the other. Additionally, the compounds of the present disclosure can be provided as a substantially pure stereoisomers, diastereomers and optical stereoisomers (such as epimers).

In some embodiments, the compounds of the present disclosure, or salts thereof, are substantially isolated. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

The structures depicted herein may omit necessary hydrogen atoms to complete the appropriate valency. Thus, in some instances a carbon atom or nitrogen atom may appear to have an open valency (i.e., a carbon atom with only two bonds showing would implicitly also be bonded to two hydrogen atoms; in addition, a nitrogen atom with a single bond depicted would implicitly also be bonded to two hydrogen atoms). For example, “—N” would be considered by one skilled in the art to be “—NH₂.” Thus, in any structure depicted herein wherein a valency is open, one or more hydrogen atoms, as appropriate, is implicit, and is only omitted for brevity.

It is to be understood that the amounts of any of the reactant compounds described herein are merely exemplary and may be increased or decreased by the skilled artisan according the particular needs of the artisan. In some embodiments, the molar ratios of any one or more of the reactants is conserved even though the absolute amounts may be altered.

The present disclosure provides methods of preparing Prurisol™ ((−) cis-[4-[2-amino-6-(cyclopropylamino)-9H-purin-9-yl]-2-cyclopentene-1-hydroxymethyl acetate). In some embodiments, Prurisol™ ((−) cis-[4-[2-amino-6-(cyclopropylamino)-9H-purin-9-yl]-2-cyclopentene-1-hydroxymethyl acetate) is a compound having the formula:

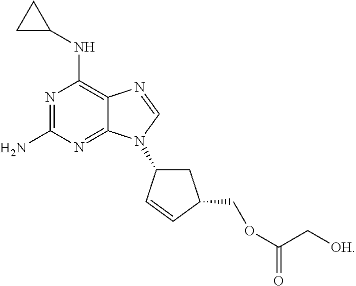 [SMILES: Nc1nc(NC2CC2)c2ncn([C@H]3C=C[C@@H](COC(=O)CO)C3)c2n1]

In some embodiments, the method comprises:

a) adding 1-ethyl-3-(3-dimethylaminopropyl)carbodiimide (EDC-HCl) to a suspension comprising Abacavir free base, a coupling agent, and 4-dimethylaminopyridine (DMAP) in a solvent under conditions sufficient to produce ((1S, 4R)-4-(2-amino-6-(cyclopropylamino)-9H-purin-9-yl) cyclopent-2-en-1-yl)methyl 2-(tert-butoxy) acetate; and

b) adding a strong acid to a solution of ((1S, 4R)-4-(2-amino-6-(cyclopropylamino)-9H-purin-9-yl) cyclopent-2-en-1-yl)methyl 2-(tert-butoxy) acetate in a solvent under conditions sufficient to produce Prurisol™ ((−) cis-[4-[2-amino-6-(cyclopropylamino)-9H-purin-9-yl]-2-cyclopentene-1-hydroxymethyl acetate).

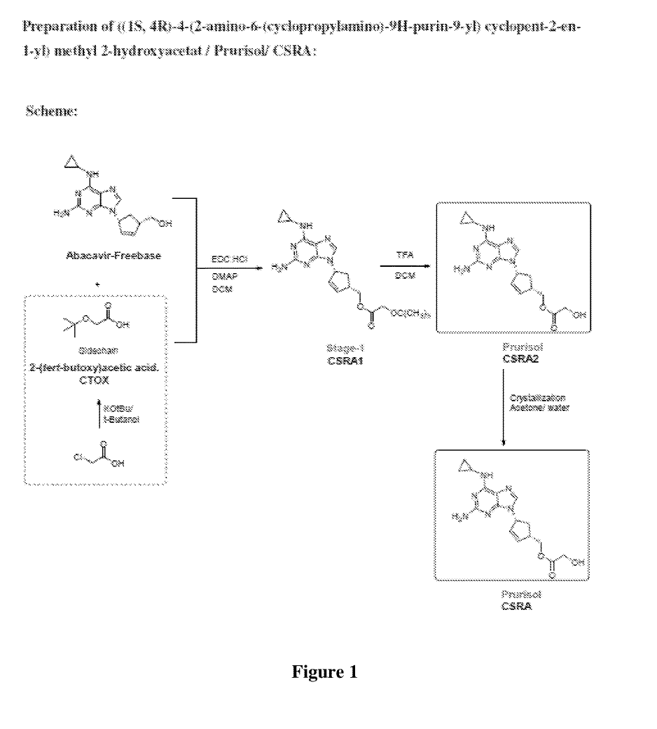

Generally, in the first step (see, FIG. 1), 1-ethyl-3-(3-dimethylaminopropyl) carbodiimide (EDC-HCl) is added to a suspension comprising Abacavir free base, a coupling agent, and 4-dimethylaminopyridine (DMAP) in a solvent under conditions sufficient to produce ((1S, 4R)-4-(2-amino-6-(cyclopropylamino)-9H-purin-9-yl)cyclopent-2-en-1-yl)methyl 2-(tert-butoxy) acetate.

In some embodiments, the solvent is dichloromethane (DCM) or tetrahydrofuran (THF). In some embodiments, the solvent is DCM. In some embodiments, it may be desired that the water content of the DCM be no more than about 0.10% (w/v determined by KF). In some embodiments, the solvent, such as DCM (e.g., 750 mL), is added to a reaction chamber, such as a round bottom flask, at about 25° C. to about 30° C.

In some embodiments, Abacavir (e.g., 75 g) is added to the reaction chamber containing the DCM at about 25° C. to about 30° C.

In some embodiments, the coupling agent is 2-(tert-butoxy) acetic acid (CTOX), (benzotriazol-1-yloxy)tripyrrolidinophosphonium hexafluorophosphate (PyBOP), or [dimethylamino(triazolo[4,5-b]pyridin-3-yloxy)methylidene]-dimethylazanium hexafluorophosphate (HATU), or any combination thereof. In some embodiments, the coupling agent is CTOX. In some embodiments, the coupling agent, such as CTOX (e.g., 41.54 g), is added to the reaction chamber at about 25° C. to about 30° C. In some embodiments, the reaction chamber is flushed with DCM (e.g., 75 mL), and stirred for about 10 minutes to about 20 minutes at about 25° C. to about 30° C.

In some embodiments, DMAP (e.g., 48 g) is added to the reaction chamber containing the DCM, Abacavir, and coupling agent at about 25° C. to about 30° C. The reaction mass may become a thick slurry followed by a free slurry. In some embodiments, the reaction mass is cooled to about 25° C. to about 30° C. and stirred for about 5 minutes to about 10 minutes.

In some embodiments, EDC-HCl (e.g., 25.11 g) is added to the reaction chamber containing the DCM, Abacavir, coupling agent, and DMAP under a nitrogen atmosphere at about 25° C. to about 30° C., and stirred at about 25° C. to about 30° C. for about 30 minutes to 60 minutes. This process of adding EDC-HCl can be repeated once (e.g., 25.11 g) or twice more (e.g., 25.11 g and 25.11 g each), with a final stirring at about 25° C. to about 30° C. for about 2 hours to about 3 hours.

At this point, a sample can be analyzed for Abacavir content by, for example, HPLC. A value of no more than 3.0% (% area) is desired. If the above reaction process is complete, the reaction can proceed to the addition of the 10% ammonium chloride solution or a brine solution as described below. If the reaction is not complete, the reaction can be allowed to proceed an additional one hour to three hours.

After three additional hours of reaction, if unreacted Abacavir is 3% to 7%, then the following steps can be carried out. CTOX (e.g., 3.46 g) can be added to the reaction chamber at about 25° C. to about 30° C. DMAP (e.g., 3.2 g) can be added to the reaction chamber at about 25° C. to about 30° C. The reaction mass can be stirred at about 25° C. to about 30° C. for about 5 minutes to about 10 minutes. EDC-HCl (e.g., 5.02 g) can be added to the reaction chamber under nitrogen atmosphere at about 25° C. to about 30° C. The reaction mass can be stirred at about 25° C. to about 30° C. for about 45 minutes to about 60 minutes. At this point, another sample can be analyzed for Abacavir content by, for example, the method described above. If the above reaction process is complete (i.e., a value of no more than 3.0% is obtained), the reaction can proceed to the addition of the 10% ammonium chloride solution or a brine solution as described below. If the reaction is still not complete, the process can be completed until the desired extent of the reaction is obtained.

After three additional hours of reaction, if unreacted Abacavir is greater than 7%, then the following steps can be carried out. CTOX (e.g., 6.92 g) can be added to the reaction chamber at about 25° C. to about 30° C. DMAP (e.g., 6.4 g) can be added to the reaction chamber at about 25° C. to about 30° C. The reaction mass can be stirred at about 25° C. to about 30° C. for about 5 minutes to about 10 minutes. EDC-HCl (e.g., 10.04 g) can be added to the reaction chamber under nitrogen atmosphere at about 25° C. to about 30° C. The reaction mass can be stirred at about 25° C. to about 30° C. for about 45 minutes to about 60 minutes. At this point, another sample can be analyzed for Abacavir content by, for example, the method described above. If the above reaction process is complete (i.e., a value of no more than 3.0% is obtained), the reaction can proceed to the addition of the 10% ammonium chloride solution or a brine solution as described below. If the reaction is still not complete, the process can be completed until the desired extent of the reaction is obtained.

In some embodiments, the 10% ammonium chloride solution can be prepared by, for example, adding water (e.g., 375 mL) and ammonium chloride (e.g., 37.5 g) at about 20° C. to about 30° C., and stirring until a clear solution is produced. In some embodiments, the prepared 10% ammonium chloride solution or a brine solution is added to the reaction mixture at about 20° C. to about 30° C. In some embodiments, the reaction mixture is stirred for about 20 minutes to about 30 minutes at about 20° C. to about 30° C. In some embodiments, the reaction mixture is allowed to settle for about 20 minutes to about 30 minutes. In some embodiments, the bottom organic layer (which contains the product) is separated from the top aqueous layer. In some embodiments, the organic layer is concentrated under vacuum (at less than 40° C., and at no less than 600 mm of Hg at the end of the distillation) to, for example, less than 2.0 volumes, to produce a crude ((1S, 4R)-4-(2-amino-6-(cyclopropylamino)-9H-purin-9-yl)cyclopent-2-en-1-yl)methyl 2-(tert-butoxy) acetate reaction product (which is a liquid). The vacuum can be released at this point by nitrogen.

In some embodiments, the crude reaction product is dissolved in toluene (e.g., 150 mL) at less than about 40° C. In some embodiments, traces of DCM are removed by distillation vacuum at less than about 40° C. (at not less than 600 mm of Hg) until less than 2.0 volumes is achieved. The vacuum is released with nitrogen and additional toluene (e.g., 675 mL) is added to the distilled mass, which is stirred for about 15 minutes to about 30 minutes at about 25° C. to about 35° C. until a clear solution is obtained. In some embodiments, the reaction mass is cooled to about 5° C. to about 15° C. In some embodiments, the crude reaction product is further washed with 15% aqueous acetic acid, washed with water, washed with a 5% aqueous sodium bicarbonate solution, and washed with a 10% aqueous sodium chloride solution.

The 15% acetic acid solution can be prepared by, for example, adding water (e.g., 319 mL) into a reaction chamber at about 25° C. to about 30° C. Acetic acid (e.g., 56 mL) is added to the water in the reaction chamber at about 25° C. to about 30° C., and stirred for about 10 minutes to about 15 minutes at about 25° C. to about 30° C. In some embodiments, the prepared 15% acetic acid solution is added to the reaction mass at about 5° C. to about 15° C., and stirred for about 20 minutes to about 30 minutes at about 5° C. to about 15° C. In some embodiments, the mixture is allowed to settle for about 20 minutes to about 30 minutes. In some embodiments, the bottom aqueous layer is separated from the top organic layer(which contains the product). In some embodiments, the aqueous layer can be used for back extraction as described below.

In some embodiments, the reaction mass is washed again with a 15% acetic acid solution. For example, in some embodiments, water (e.g., 375 mL) is added to the organic layer at about 10° C. to about 30° C., and stirred for about 20 minutes to about 30 minutes. In some embodiments, the mixture is allowed to settle for about 20 minutes to about 30 minutes, and the bottom aqueous layer is separated from the top organic layer. In some embodiments, the organic layer is analyzed for impurities by, for example, HPLC analysis. A desired impurity at 0.83 RRT is not more than 0.10%. If the desired level of impurity is not achieved, the following procedure can be carried out. Another 15% acetic acid solution can be prepared by, for example, adding water (e.g., 191 mL) into a reaction chamber at about 25° C. to about 30° C. Acetic acid (e.g., 34 mL) is added to the reaction chamber containing the water at about 25° C. to about 30° C., and stirred for about 10 minutes to about 15 minutes at about 25° C. to about 30° C. The prepared 15% acetic acid solution is added to the organic layer at about 5° C. to about 15° C., and stirred for about 5 minutes to about 10 minutes at about 5° C. to about 15° C. In some embodiments, the mixture is allowed to settle for about 20 minutes to about 30 minutes. In some embodiments, it may be desired to analyze the bottom aqueous layer sample for pH. A desired pH of the aqueous layer is no more than about 3.0.

If a desired pH is not obtained, then the bottom aqueous layer is separated from the top organic layer, and the following procedure can be carried out. Another 15% acetic acid solution can be prepared by, for example, adding water (e.g., 127.5 mL) into a reaction chamber at about 25° C. to about 30° C. Acetic acid (e.g., 22.5 mL) is added to the reaction chamber containing the water at about 25° C. to about 30° C., and stirred for about 10 minutes to about 15 minutes at about 25° C. to about 30° C. The prepared 15% acetic acid solution is added to the organic layer at about 5° C. to about 15° C., and stirred for about 10 minutes to about 20 minutes at about 5° C. to about 15° C. In some embodiments, the mixture is allowed to settle for about 20 minutes to about 30 minutes. This process can be repeated, as necessary until a desired pH of the aqueous layer is achieved. The aqueous layers can be saved for future back extraction as described below.

If the desired level of impurity or pH is not achieved, the following back extraction procedure can be carried out using the combined aqueous layers. In some embodiments, toluene (e.g., 300 mL) is added to the above combined aqueous layers, and stirred for about 20 minutes to about 30 minutes. In some embodiments, the mixture is allowed to settle for about 20 minutes to about 30 minutes, and the bottom aqueous layer is separated from the top organic layer (which contains the product).

If a desired impurity level or pH has been obtained, the previous organic layers from the above procedures can be combined. In some embodiments, water (e.g., 375 mL) is added to the above organic layer, and stirred for about 20 minutes to about 30 minutes. In some embodiments, the mixture is allowed to settle for about 20 minutes to about 30 minutes, and the bottom aqueous layer is separated from the top organic layer(which contains the product). In some embodiments, the organic layer can be analyzed for impurity by, for example, HPLC. A desired level of impurity at 0.83RRT is no more than about 0.10%.

If the desired level of impurity is not obtained, then the following procedures can be carried out. For example, in some embodiments, a 15% acetic acid solution can be prepared by adding water (e.g., 191 mL) into a reaction chamber at about 25° C. to about 30° C., and further adding acetic acid (e.g., 34 mL) at about 25° C. to about 30° C., and stirred for about 10 minutes to about 15 minutes at about 25° C. to about 30° C. The prepared 15% acetic acid solution is added to the organic layer at about 5° C. to about 15° C., and stirred for about 20 minutes to about 30 minutes at about 5° C. to about 15° C. In some embodiments, the mixture is allowed to settle for about 20 minutes to about 30 minutes, and the bottom aqueous layer is separated from the top organic layer. Water (e.g., 375 mL) is again added to the organic layer, and stirred for about 20 minutes to about 30 minutes. In some embodiments, the mixture is allowed to settle for about 20 minutes to about 30 minutes, and the bottom aqueous layer is separated from the top organic layer (which contains the product). The aqueous layer is saved. In some embodiments, the organic layer is analyzed for impurities by, for example, the procedures described above. Again, a desired impurity limit is no more than about 0.10%. If the desired impurity level is not achieved, the preceding steps can be repeated as necessary until the desired level of impurity is obtained.

If the desired level of impurity of the organic layer is obtained, then the following procedures can be carried out. Water (e.g., 375 mL) is added to the organic layer, and stirred for about 20 minutes to about 30 minutes at about 10° C. to about 30° C. In some embodiments, the mixture is allowed to settle for about 20 minutes to about 30 minutes, and the bottom aqueous layer is separated from the top organic layer (which contains the product). Water (e.g., 375 mL) is added to the organic layer, and stirred for about 20 minutes to about 30 minutes at about 20° C. to about 30° C. In some embodiments, the mixture is allowed to settle for about 20 minutes to about 30 minutes, and the bottom aqueous layer is separated from the top organic layer (which contains the product).

In some embodiments, the reaction mass is further washed with a 6% sodium bicarbonate solution. For example, in some embodiments, the 6% sodium bicarbonate solution can be prepared by adding water (e.g., 300 mL) and sodium bicarbonate (e.g., 18 g) to a reaction chamber at about 20° C. to about 30° C., and stirred at about 20° C. to about 30° C. until a clear solution is obtained. The prepared 6% sodium bicarbonate solution is added to the organic layer at about 20° C. to about 30° C., and stirred for about 5 minutes to about 10 minutes, and allowed to settle for about 20 minutes to about 30 minutes. In some embodiments, it may be desired to analyze the pH of the bottom aqueous layer sample. A desired pH for the aqueous layer is no less than 7.0.

If the desired pH is not obtained, the following procedures can be carried out. Another 5% sodium bicarbonate solution can be prepared by adding water (e.g., 150 mL) and sodium bicarbonate (e.g., 9 g) to a reaction chamber at about 20° C. to about 30° C., and stirring at about 20° C. to about 30° C. until a clear solution is obtained. The prepared 5% sodium bicarbonate solution is added to the organic layer. In some embodiments, a sample of the bottom aqueous layer can be analyzed for pH as described above. If the desired pH is not obtained, the previous steps can be repeated as necessary until the desired pH is obtained.

If the desired pH is obtained, the following procedures can be carried out. In some embodiments, the mixture is stirred for about 20 minutes to about 30 minutes at about 20° C. to about 30° C. In some embodiments, the mixture is allowed to settle for about 20 minutes to about 30 minutes, and the bottom aqueous layer is separated from the top organic layer (which contains the product). If an emulsion is observed, the emulsion along with the aqueous layer can be collected. In some embodiments, the aqueous layer is added back into reaction chamber from the previous step. In some embodiments, toluene (e.g., 150 mL) is added to the aqueous layer, and stirred for about 20 minutes to about 30 minutes. In some embodiments, the mixture is stirred for about 20 minutes to about 30 minutes, and the bottom aqueous layer is separated from the top organic layer (which contains the product). If an emulsion is observed, the emulsion along with the aqueous layer can be collected.

In some embodiments, the reaction mass is further washed with a 10% sodium chloride solution. For example, in some embodiments, the 10% sodium chloride solution is prepared by adding water (e.g., 150 mL) and sodium chloride (e.g., 15 g) in a reaction chamber at about 20° C. to about 30° C., and stirred at about 20° C. to about 30° C. until a clear solution is obtained. The prepared 10% sodium chloride solution is added to the organic layer, and stirred for about 20 minutes to about 30 minutes at about 20° C. to about 30° C. In some embodiments, the mixture is stirred for about 20 minutes to about 30 minutes, and the bottom aqueous layer is separated from the top organic layer (which contains the product). If an emulsion is observed, the emulsion along with the aqueous layer can be collected. In some embodiments, the reaction mass is further washed once, twice, or three times again with the 10% sodium chloride solution as described above. In some embodiments, the organic layer is filtered through a micron filter. In some embodiments, the organic layer is distilled under vacuum (at no less than about 650 mm of Hg) until the inside volume is less than 1.5 volumes at less than 60° C.

In some embodiments, the organic layer is concentrated under vacuum. In some embodiments, the organic layer is dissolved in DCM (e.g., 150 mL) or THF, and stirred for about 15 minutes to about 30 minutes to obtain a clear solution at about 20° C. to about 30° C. In some embodiments, n-heptane (e.g., 450 mL) or n-hexane is slowly added over a period of 60 minutes to 120 minutes, and the suspension is stirred at about 25° C. to about 30° C. for about 10 hours to about 12 hours. In some embodiments, the suspension cooled to about 0° C. to about 5° C., and further stirred at about 0° C. to about 5° C. for about 4 to about 6 hours.

In some embodiments, the resulting product is filtered, and washed with n-heptane (e.g., 150 mL) or n-hexane. In some embodiments, the product is further dried under vacuum (at no less than about 650 mm of Hg) at about 50° C. to about 55° C. for about 8 hours to about 10 hours. In some embodiments, it may be desired to analyze a sample of the product for loss on drying (LOD) at about 80° C. by, for example, HMA for 30 minutes (% w/w). A desired LOD value is no more than about 4.0%. The product can be unloaded and stored at less than 25° C. The resultant product is ((1S, 4R)-4-(2-amino-6-(cyclopropylamino)-9H-purin-9-yl)cyclopent-2-en-1-yl)methyl 2-(tert-butoxy) acetate.

Generally, in the second step (see, FIG. 1), a strong acid is added to a solution of ((1S, 4R)-4-(2-amino-6-(cyclopropylamino)-9H-purin-9-yl) cyclopent-2-en-1-yl)methyl 2-(tert-butoxy) acetate in a solvent under conditions sufficient to produce Prurisol™ ((−) cis-[4-[2-amino-6-(cyclopropylamino)-9H-purin-9-yl]-2-cyclopentene-1-hydroxymethyl acetate). In some embodiments, the strong acid is trifluoro acetic acid (TFA).

In particular, in some embodiments, DCM (e.g., 350 mL) is added to a reaction chamber at about 25° C. to about 30° C. In some embodiments, a DCM sample is analyzed for water content by, for example, KF (% w/v). A desired DCM water content is no more than about 0.10 (% w/v). If the desired DCM water content is not obtained, the preceding DCM treatment can be repeated using 7.0 volumes of DCM until the desired DCM water content is 30 achieved. If IPC complies follow below operation onwards.

In some embodiments, ((1S, 4R)-4-(2-amino-6-(cyclopropylamino)-9H-purin-9-yl) cyclopent-2-en-1-yl)methyl 2-(tert-butoxy) acetate (e.g., 50 g) is added to the reaction chamber at about 25° C. to about 30° C. under stirring. The reaction chamber is flushed with DCM (e.g., 25 mL) at about 25° C. to about 30° C., and stirred for about 15 minutes to about 30 minutes to obtain a clear solution. If a clear solution is not observed, the stirring can continue until a clear solution is obtained. In some embodiments, the reaction chamber is flushed with nitrogen to ensure any atmospheric moisture is replaced with nitrogen. In some embodiments, the reaction mass is cooled to about 0° C. to about 5° C.

In some embodiments, the strong acid is added to the solution of ((1S, 4R)-4-(2-amino-6-(cyclopropylamino)-9H-purin-9-yl) cyclopent-2-en-1-yl)methyl 2-(tert-butoxy) acetate in DCM at about 0° C. to about 5° C. In particular, TFA (e.g., 250 g/168 mL) is added slowly to the reaction chamber at about 0° C. to about 10° C. under stirring. In some embodiments, the reaction chamber is flushed with DCM (e.g., 25 mL) at about 25° C. to about 30° C. In some embodiments, the temperature is increased slowly to about 25° C. to about 30° C.

In some embodiments, the solution is further stirred at about 25° C. to about 30° C. for about 4 hours to about 6 hours. In some embodiments, it may be desired to analyze the solution sample for ((1S, 4R)-4-(2-amino-6-(cyclopropylamino)-9H-purin-9-yl) cyclopent-2-en-1-yl)methyl 2-(tert-butoxy) acetate content by, for example, HPLC. A desired ((1S, 4R)-4-(2-amino-6-(cyclopropylamino)-9H-purin-9-yl) cyclopent-2-en-1-yl)methyl 2-(tert-butoxy) acetate content is no more than about 0.5%. If the desired ((1S, 4R)-4-(2-amino-6-(cyclopropylamino)-9H-purin-9-yl) cyclopent-2-en-1-yl)methyl 2-(tert-butoxy) acetate content is not obtained, the sample can be analyzed about every 2.0 hours until the desired content is obtained. In some embodiments, the reaction chamber is flushed with nitrogen to ensure that any atmospheric moisture is replaced with nitrogen.

In some embodiments, the reaction mass is cooled to about −10° C. to about 0° C. In some embodiments, triethylamine (TEA) (e.g., 250 g) is added at a temperature less than about 20° C. In some embodiments, the solution is stirred for about 15 minutes to about 30 minutes at about 0° C. to about 20° C. In some embodiments, it may be desired to analyze a sample for pH. A desired pH of the reaction mass after TEA quenching is no less than about 7.0.

If the desired pH is not obtained, the following procedures can be carried out. TEA (e.g., 25 g) is added slowly to the solution at less than about 20° C., and stirred for about 15 minutes to about 30 minutes at about 0° C. to about 20° C. A sample can be analyzed for pH as described above. Again, a desired pH of the reaction mass after TEA quenching is no less than about 7.0. If the desired pH is not obtained, the following procedures can be repeated as necessary until the desired pH is obtained.

If the desired pH is obtained, the following procedures can be carried out. In some embodiments, the reaction mass is concentrated under vacuum (at no less than about 400 mm of Hg) until the inside volume is 6-10 volumes (w.r.t batch size) or up to about 50% at less than about 40° C. In some embodiments, the reaction mass is cooled to about 20° C. to about 30° C. In some embodiments, water (e.g., 500 mL) is further added, and the reaction mass is stirred at about 20° C. to about 30° C. It is noted that the reaction mass is a biphasic, solid product that floats on the top of the aqueous layer during reaction mass settling. In some embodiments, the product is filtered and spray washed with water (e.g., 150 mL). In some embodiments, the material is unloaded and the slurry washed with water (e.g., 250 mL) at about 20° C. to about 30° C. In some embodiments, the product is filtered and spray washed with water (e.g., 150 mL), and suck or spin dried until the absence of the mother liquor is obtained. In some embodiments, the material is unloaded.

In some embodiments, the material slurry washed with water is wetted with acetone and dried under vacuum to produce Prurisol™ ((−) cis-[4-[2-amino-6-(cyclopropylamino)-9H-purin-9-yl]-2-cyclopentene-1-hydroxymethyl acetate). In particular, in some embodiments, acetone (e.g., 250 mL) is added to a reaction chamber, which is cooled to about 0° C. to about 10° C. The wet material from the previous reaction is added to the reaction chamber containing the acetone, and stirred for about 30 minutes to about 60 minutes at about 0° C. to about 10° C. If the reaction mass is a thick slurry and not stirrable, additional acetone (e.g., 150 mL) can be added. In some embodiments, the solid product is filtered at about 0° C. to about 10° C. In some embodiments, the above wet cake is washed with acetone (e.g., 100 mL), and suck or spin dried until the absence of the mother liquor is obtained. In some embodiments, the material is unloaded. In some embodiments, it may desired to analyze a sample of the wet sample for related substances by, for example, HPLC (% area). A desired Abacavir content is no more than about 0.40. A desired ((1S, 4R)-4-(2-amino-6-(cyclopropylamino)-9H-purin-9-yl) cyclopent-2-en-1-yl)methyl 2-(tert-butoxy) acetate content is no more than about 0.20. A desired any individual impurity content is no more than about 0.15. A desired total impurity content is no more than about 1.0.

If the desired impurity content is not obtained, the following procedures can be carried out. In some embodiments, acetone (e.g., 315 mL) and water (e.g., 35 mL) are added to a reaction chamber. The wet material from the previous procedure is added to the reaction chamber, and heated to a mild reflux for about 4 hours to about 10 hours. In some embodiments, the reaction is cooled to about 20° C. to about 30° C. slowly in no less than about 1.0 hours. The reaction is stirred for about 1 hour to about 2 hours at about 20° C. to about 30° C., and the product is filtered at about 20° C. to about 30° C. In some embodiments, the wet cake is washed with acetone (e.g., 150 mL), and suck or spin dried until the absence of the mother liquor is obtained. In some embodiments, it may desired to analyze a sample of the dried product for related substances by, for example, HPLC (% area). A desired Abacavir content, ((1S, 4R)-4-(2-amino-6-(cyclopropylamino)-9H-purin-9-yl) cyclopent-2-en-1-yl)methyl 2-(tert-butoxy) acetate content, individual impurity content, and total impurity content is set forth above. If the desired impurity content is not obtained, the preceding procedures can be carried out until the desired impurity content is obtained.

If the desired impurity content is obtained, the following procedures can be carried out. In some embodiments, the material is dried under vacuum (at no less than about 650 mm of Hg) at about 45° C. to about 50° C. for about 8 hours to about 12 hours. In some embodiments, it may desired to analyze a sample of the dried product for water content by, for example, KF (% w/w). A desired water content is no more than about 5.5. If the desired water content is not obtained, the drying can continue under vacuum (at no less than about 650 mm of Hg) at about 45° C. to about 50° C., whereby the sample can be analyzed for water content at about every 3.0 hours until the desired water content is achieved. In some embodiments, it may desired to analyze a sample of the dried product for acetone content by, for example, GC-HS. A desired acetone content is no more than about 5%. If the desired acetone content is not obtained, the drying can continue under vacuum (at no less than about 650 mm of Hg) at about 45° C. to about 50° C., whereby the sample can be analyzed for acetone content at about every 3.0 hours until the desired acetone content is achieved. Upon achieving a desired water and/or acetone content, the dried material can be unloaded.

In some embodiments, the methods of preparing Prurisol™ ((−) cis-[4-[2-amino-6-(cyclopropylamino)-9H-purin-9-yl]-2-cyclopentene-1-hydroxymethyl acetate)described herein further comprise a crystallization process. For example, Prurisol™ ((−) cis-[4-[2-amino-6-(cyclopropylamino)-9H-purin-9-yl]-2-cyclopentene-1-hydroxymethyl acetate) is dissolved in a mixture of acetone and water under conditions sufficient to produce crystallized Prurisol™ ((−) cis-[4-[2-amino-6-(cyclopropylamino)-9H-purin-9-yl]-2-cyclopentene-1-hydroxymethyl acetate). In some embodiments, the Prurisol™ ((−) cis-[4-[2-amino-6-(cyclopropylamino)-9H-purin-9-yl]-2-cyclopentene-1-hydroxymethyl acetate) solid is dissolved in mixture of acetone and water at about 50° C. to about 55° C. and is cooled to about 0° C. to about 5° C. In some embodiments, the product is filtered, washed with acetone, and dried under vacuum to produce crystallized Prurisol™ ((−) cis-[4-[2-amino-6-(cyclopropylamino)-9H-purin-9-yl]-2-cyclopentene-1-hydroxymethyl acetate).

In particular, in some embodiments, acetone (e.g., 60 mL) is added into a reaction chamber, and the contents are heated to reflux and maintained for about 30 minutes. In some embodiments, the contents are cooled to about 25° C. to about 35° C. and unloaded. Acetone (e.g., 252 mL) is again added to the reaction chamber without stirring under a nitrogen atmosphere at about 25° C. to about 35° C. Water (e.g., 108 mL) is added to the reaction chamber under nitrogen atmosphere at about 25° C. to about 35° C. Uncrystallized Prurisol™ ((−) cis-[4-[2-amino-6-(cyclopropylamino)-9H-purin-9-yl]-2-cyclopentene-1-hydroxymethyl acetate) (e.g., 20 g) is added to the reaction chamber at about 25° C. to about 35° C. under nitrogen atmosphere and stirred for about 10 minutes. In some embodiments, the reaction mass is heated to about 50° C. to about 55° C. The reaction mass is maintained for about 20 minutes to about 40 minutes and checked for dissolution. If the reaction mass is not a clear solution, it can be further maintained for about 3 minutes and re-checked for dissolution. In some embodiments, the reaction mass is filtered through one or more micron filters and transferred into a clean reaction chamber. In some embodiments, the clean room containing the reaction chamber is set at a temperature of about 60° C.

In some embodiments, acetone (e.g., 28 mL) is added to the reaction chamber at about 25° C. to about 35° C. Water (e.g., 12 mL) is added to the reaction chamber at about 25° C. to about 35° C., and the reaction mass is heated to about 50° C. to about 55° C. In some embodiments, the above pre-heated aqueous acetone solution is filtered through a micron filter and transferred to the clean room reaction chamber, and flushed with N₂ after washing. The temperature of the clean room reaction chamber is adjusted to about 50° C. to about 55° C. and checked for dissolution. If the reaction mass is not a clear solution, the reaction mass can be maintained for about 30 minutes and re-checked for dissolution.

The reaction mass can be maintained for about 20 minutes to about 40 minutes at about 50° C. to about 55° C. In some embodiments, the reaction mass is cooled to about 37.7° C. to about 42.5° C. in no less than about 30 minutes. In some embodiments, the reaction mass is further cooled to about 22.5° C. to about 27.5° C. in no less than about 1 hour. In some embodiments, the reaction mass is further cooled to about 12.5° C. to about 17.5° C. in no less than about 30 minutes. In some embodiments, the reaction mass is further cooled to about 0° C. to about 5° C. in no less than about 30 minutes. In some embodiments, the reaction mass is maintained at about 0° C. to about 5° C. for about 1 hour to about 2 hours. In some embodiments, the reaction mass is filtered, while maintaining the reaction mass at about 0° C. to about 5° C. in the reaction chamber.

In some embodiments, the reaction mass is washed with acetone (e.g., 40 mL) at about 25° C. to about 35° C. under nitrogen. The reaction mass is suck or spin dried for about 1 hour to about 2 hours, and the material is unloaded from the filter. In some embodiments, a sample of the reaction mass is analyzed for purity. In some embodiments, it is desired that the total impurity content is no more than about 1.0%. In some embodiments, it is desired that the Abacavir content is no more than about 0.5%. In some embodiments, it is desired that the content of any individual impurity is no more than about 0.15%.

If the desired purity is not obtained, the following procedures can be carried out. In some embodiments, acetone (e.g., 126 mL) is added to a clean room reaction chamber at about 25° C. to about 35° C. Water (e.g., 14 mL) is added to the clean room reaction chamber at about 25° C. to about 35° C. To the reaction chamber is added the reaction mass from the filter set forth above at about 25° C. to about 35° C. The reaction mass is heated to about 45° C. to about 55° C., and maintain for about 3 hours to about 5 hours. In some embodiments, the reaction mass is cooled to about 22.5° C. to about 27.5° C., and maintained for about 45 minutes to about 75 minutes. In some embodiments, the reaction mass is filtered. During the filtration process, the reaction mass is maintained at about 20° C. to about 30° C. in the reaction chamber. In some embodiments, the reaction mass is washed with acetone (e.g., 40 mL) under nitrogen, and suck dried for about 1 hour to about 2 hours. In some embodiments, the material is unloaded from the filter. In some embodiments, a sample of the reaction mass is analyzed for purity. In some embodiments, it is desired that the total impurity content is no more than about 1.0%. In some embodiments, it is desired that the Abacavir content is no more than about 0.5%. In some embodiments, it is desired that the content of any individual impurity is no more than about 0.15%. If the desired purity is not obtained, the preceding steps can be repeated until the desired purity is obtained.

If the desired purity is not obtained, the following procedures can be carried out. In some embodiments, the material is loaded uniformly into a dryer, and the product is dried for about 1 hour to about 2 hours under vacuum at no less than about 650 mm of Hg at about 25° C. to about 35° C. The hot water temperature of the dryer is increased slowly to no more than about 60° C. (i.e., the temperature of the dryer is increased to about 40° C. to about 50° C.) and dried for about 4 hours under vacuum at no less than about 650 mm of Hg. In some embodiments, the dryer is cooled to about 25° C. to about 35° C., and the vacuum is released with nitrogen. In some embodiments, the lumps in the material are broken down.

In some embodiments, the hot water temperature of the dryer is slowly increased to no more than about 70° C. (i.e., the temperature of the dryer is increased to about 55° C. to about 60° C.) and dried for about 10 hours under vacuum at no less than about 650 mm of Hg. In some embodiments, the dryer is cooled to about 25° C. to about 35° C., and the vacuum is released with nitrogen. In some embodiments, the sample is analyzed for water content. A desired water content is no more than about 6.0% w/w.

If the desired water content is not obtained, the following procedures can be carried out. In some embodiments, the hot water temperature of the dryer is slowly increased to no more than about 70° C. (i.e., the temperature of the dryer is increased to about 55° C. to about 60° C.). The sample can be analyzed for about every 4 hours until the desired water content is obtained.

If the desired water content is not obtained, the following procedures can be carried out. In some embodiments, the sample is also analyzed for acetone and DCM content by, for example, GCHS. A desired acetone content is no more than about 4300 ppm. A desired DCM content is no more than about 480 ppm. If the desired acetone content and/or DCM content is not obtained, the hot water temperature of the dryer can be slowly increased to no more than about 70° C. (i.e., the temperature of the dryer is increased to about 55° C. to about 60° C.). The sample can be analyzed for about every 4 hours until the desired acetone and/or DCM content is obtained.

In some embodiments, the product is cooled to about 25° C. to about 35° C., and the vacuum is released with nitrogen. In some embodiments, the material is unloaded from the dryer. In some embodiments, the material is sieved. The final product is crystallized Prurisolo™ ((−) cis-[4-[2-amino-6-(cyclopropylamino)-9H-purin-9-yl]-2-cyclopentene-1-hydroxymethyl acetate).

The present disclosure also provides methods of preparing CTOX. The methods of preparing CTOX can be used to prepare the CTOX used in any of the methods of preparing Prurisol™ ((−) cis-[4-[2-amino-6-(cyclopropylamino)-9H-purin-9-yl]-2-cyclopentene-1-hydroxymethyl acetate) described herein. In some embodiments, a haloacetic acid is added to a suspension of t-butanol and potassium tert-butoxide. In some embodiments, the water content of the t-butanol is no more than 0.20%.

In particular, in some embodiments, t-butanol (e.g., 1200 mL) is added to a reaction chamber at about 30° C. to about 40° C. In some embodiments, the potassium tert-butoxide (e.g., 262 g) is added to the reaction chamber containing the t-butanol at about 30° C. to about 50° C. In some embodiments, the reaction chamber is flushed with t-butanol (e.g., 50 mL) at a temperature greater than about 30° C.

In some embodiments, the haloacetic acid (e.g., 100 g) is added to the suspension of the t-butanol and potassium tert-butoxide slowly at about 30° C. to about 50° C. In some embodiments, the haloactic acid is chloroacetic acid, bromoacetic acid, or iodoacteic acid. In some embodiments, the reaction chamber is flushed with t-butanol (e.g., 50 mL) at a temperature above about 30° C.

In some embodiments, the reaction mass of the haloacetic acid, t-butanol, and potassium tert-butoxide is heated to reflux and maintained at about 80° C. to about 85° C. for about 6 hours to about 8 hours. In some embodiments, the reaction mass is maintained at about 79° C. to about 85° C. for about 6 hours to about 8 hours. In some embodiments, the reaction mass is then cooled to about 30° C. to about 45° C. At this point, the yield and purity can be determined by routine procedures. A desired yield can be no less than 45% and GC purity can be no less than 97%. If undesired results are obtained, the reaction mass can be further refluxed at about 79° C. to about 85° C. for about 3 additional hours.

In some embodiments, water (e.g., 200 mL) is added to the reaction mass of haloacetic acid, t-butanol, and potassium tert-butoxide at about 30° C. to about 45° C., and the reaction mass is concentrated (i.e., distilled) under vacuum. In some embodiments, the vacuum is no less than about 650 mm of Hg at less than about 60° C. until the inside volume becomes less than 3.0 volumes.

In some embodiments, water (e.g., 800 mL) is added to the reaction mass at about 20° C. to about 35° C., and stirred for about 15 minutes to about 20 minutes. A clear solution is desired. If the reaction mass is not clear, the reaction mass can be further stirred. The reaction mass is then washed with methyl tert-butyl ether (MTBE) (e.g., 300 mL) at about 20° C. to about 35° C., and stirred at about 25° C. to about 35° C. for about 30 minutes to about 45 minutes. In some embodiments, the reaction mass is allowed to settle for about 30 minutes to about 45 minutes.

In some embodiments, the top organic layer is separated from the bottom aqueous layer (which contains the desired product). In some embodiments, the aqueous layer is added to a reaction chamber. In some embodiments, the product is extracted with MTBE or diethyl ether. In some embodiments, the product is extracted with MTBE (e.g., 300 mL) at about 20° C. to about 35° C., and stirred for about 30 minutes to about 45 minutes. In some embodiments, the reaction mass is allowed to settle for about 30 minutes to about 45 minutes.

In some embodiments, the top organic layer is again separated from the bottom aqueous layer (which contains the desired product). In some embodiments, the aqueous layer is added to a reaction chamber. In some embodiments, the aqueous layer is cooled to about 20° C. to about 30° C. In some embodiments, 4.0N sulfuric acid solution can be prepared by adding concentrated sulfuric acid (e.g., 55 mL) is added to pre-chilled water (e.g., 445 mL) at about 20° C. to about 30° C. The pH of the aqueous layer is adjusted to 1.5 to 3.0 using the prepared sulfuric acid solution or a hydrochloric acid solution. The aqueous layer is stirred for about 15 minutes to about 30 minutes. In some embodiments, the product is extracted with MTBE (e.g., 500 mL) or diethyl ether at about 20° C. to about 30° C., and stirred for about 30 minutes to about 45 minutes. In some embodiments, the salt product is filtered through, for example, a leaf filter. In some embodiments, the salt product is washed with MTBE (e.g., 100 mL), and the filtrate is collected and placed into a reaction chamber. The bottom aqueous layer is separated from the top organic layer (the product is present in the organic layer) and the organic layer is stored in a new reaction chamber. The aqueous layer is then added to a reaction chamber and the above process is repeated a plurality of times with MTBE (e.g., 500 mL, 500 mL, and 400 mL) at about 20° C. to about 30° C., and stirred for about 30 minutes to about 45 minutes. The top organic layer of each round is collected and combined into a reaction chamber.

In some embodiments, the organic layer is washed with an aqueous sodium chloride solution (i.e., a brine solution). In some embodiments, the brine solution can be prepared by dissolving sodium chloride (e.g., 50 g) in water (e.g., 200 mL) and adding the brine solution to a reaction chamber containing the organic layer. In some embodiments, the reaction mass is stirred at about 20° C. to about 30° C. for about 30 minutes to about 45 minutes. In some embodiments, the reaction mass is allowed to settle for about 30 minutes to about 45 minutes. In some embodiments, the bottom aqueous layer is separated from the top organic layer (which contains the product). In some embodiments, the washing step with the aqueous sodium chloride solution (i.e., a brine solution) can be repeated, wherein a second brine solution can be prepared by dissolving sodium chloride (e.g., 50 g) in water (e.g., 200 mL) and adding the brine solution to the reaction chamber containing the organic layer.

In some embodiments, the organic layer is filtered through a micron filter. In some embodiments, the organic layer is concentrated under vacuum (at no less than 650 mm of Hg at less than 40° C.) until there is an absence of a distillate. In some embodiments, the distillation under vacuum can continue until the water content is no more than 2.0%. The product produced is liquid CTOX. It is desired to have an MTBE content of no more than 4.0% and a t-butanol content of no more than 4.0%. The CTOX product can be stored at less than 25° C.

Particular embodiments of the present disclosure include, but are not limited to:

### Embodiment 1: A Method of Preparing a Compound Having the Formula

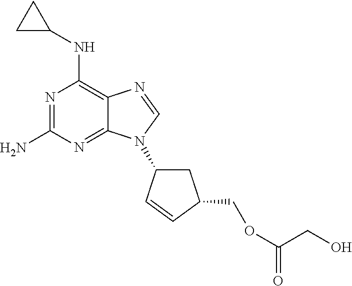 [SMILES: Nc1nc(NC2CC2)c2ncn([C@H]3C=C[C@@H](COC(=O)CO)C3)c2n1]

Prurisol™ ((−) cis-[4-[2-amino-6-(cyclopropylamino)-9H-purin-9-yl]-2-cyclopentene-1-hydroxymethyl acetate)

comprising: a) adding 1-ethyl-3-(3-dimethylaminopropyl)carbodiimide (EDC-HCl) to a suspension comprising Abacavir free base, a coupling agent, and 4-dimethylaminopyridine (DMAP) in a solvent under conditions sufficient to produce ((1S, 4R)-4-(2-amino-6-(cyclopropylamino)-9H-purin-9-yl)cyclopent-2-en-1-yl)methyl 2-(tert-butoxy) acetate; and b) adding a strong acid to a solution of ((1S, 4R)-4-(2-amino-6-(cyclopropylamino)-9H-purin-9-yl) cyclopent-2-en-1-yl)methyl 2-(tert-butoxy) acetate in a solvent under conditions sufficient to produce Prurisol™ ((−) cis-[4-[2-amino-6-(cyclopropylamino)-9H-purin-9-yl]-2-cyclopentene-1-hydroxymethyl acetate).

### Embodiment 2

The method of embodiment 1 wherein the coupling agent is 2-(tert-butoxy) acetic acid (CTOX), (benzotriazol-1-yloxy)tripyrrolidinophosphonium hexafluorophosphate (PyBOP), or [dimethylamino(triazolo [4,5 -b]pyridin-3 -yloxy)methylidene]-dimethylazanium hexafluorophosphate (HATU), or any combination thereof.

### Embodiment 3

The method of embodiment 1 wherein the solvent is dichloromethane (DCM) or tetrahydrofuran (THF).

### Embodiment 4

The method of embodiment 1 wherein the strong acid is trifluoro acetic acid (TFA).

### Embodiment 5

The method of any one of embodiments 1 to 4 further comprising: c) dissolving Prurisol™ ((−) cis-4-[2-amino-6-(cyclopropylamino)-9H-purin-9-yl]-2-cyclopentene-1-hydroxymethyl acetate) in a mixture of acetone and water under conditions sufficient to produce crystallized Prurisol™ ((−) cis-[4-[2-amino-6-(cyclopropylamino)-9H-purin-9-yl]-2-cyclopentene-1-hydroxymethyl acetate).

### Embodiment 6

The method of any one of embodiments 1 to 5 further comprising stirring the reaction mixture of EDC-HCl, Abacavir free base, coupling agent, and DMAP in solvent at about 25° C. to about 30° C. for about 2 hours to about 3 hours.

### Embodiment 7

The method of embodiment 6 further comprising adding a 10% ammonium chloride solution or a brine solution to the reaction mixture, separating the organic layer, and concentrating the organic layer under vacuum to produce a crude ((1S, 4R)-4-(2-amino-6-(cyclopropylamino)-9H-purin-9-yl)cyclopent-2-en-1-yl)methyl 2-(tert-butoxy) acetate reaction product.

### Embodiment 8

The method of embodiment 7 further comprising dissolving the crude reaction product in toluene, washing the crude reaction product with 15% aqueous acetic acid, washing the crude reaction product with water, washing the crude reaction product with a 5% aqueous sodium bicarbonate solution, and washing the crude reaction product with a 10% aqueous sodium chloride solution.

### Embodiment 9

The method of embodiment 8 further comprising concentrating the organic layer under vacuum, dissolving the organic layer in DCM or THF, slowly adding n-heptane or n-hexane, stirring the suspension at about 25° C. to about 30° C. for about 10 hours to about 12 hours, and stirring the suspension at about 0° C. to about 5° C. for about 5 to about 6 hours.

### Embodiment 10

The method of embodiment 9 further comprising filtering the product, washing the product with n-heptane or n-hexane, and drying the product under vacuum to produce (1S, 4R)-4-(2-amino-6-(cyclopropylamino)-9H-purin-9-yl)cyclopent-2-en-1-yl)methyl 2-(tert-butoxy) acetate.

### Embodiment 11

The method of any one of embodiments 1 to 10 wherein the strong acid is added to the solution of ((1S, 4R)-4-(2-amino-6-(cyclopropylamino)-9H-purin-9-yl) cyclopent-2-en-1-yl)methyl 2-(tert-butoxy) acetate in DCM at about 0° C. to about 5° C. and is stirred at about 25° C. to about 30° C. for about 4 hours to about 6 hours.

### Embodiment 12

The method of embodiment 11 further comprising cooling the reaction mass to −10° C. and adding triethyl amine below 20° C.

### Embodiment 13

The method of embodiment 12 further comprising concentrating the reaction mass up to about 50%, adding water, and stirring at about 20° C. to about 30° C.

### Embodiment 14

The method of embodiment 13 further comprising filtering the product and washing the product with water.

### Embodiment 15

The method of embodiment 14 further comprising wetting the material slurry washed with water followed by acetone and drying under vacuum to produce Prurisol™ ((−) cis-[4-[2-amino-6-(cyclopropylamino)-9H-purin-9-yl]-2-cyclopentene-1-hydroxymethyl acetate).

### Embodiment 16

The method of embodiment 15 wherein the Prurisol™ ((−) cis-[4-[2-amino-6-(cyclopropylamino)-9H-purin-9-yl]-2-cyclopentene-1-hydroxymethyl acetate) solid is dissolved in mixture of acetone and water at about 50° C. to about 55° C. and is cooled to about 0° C. to about 5° C.

### Embodiment 17

The method of embodiment 16 further comprising filtering the product, washing the product with acetone, and drying the product under vacuum to produce crystallized Prurisol™ ((−) cis-[4-[2-amino-6-(cyclopropylamino)-9H-purin-9-yl]-2-cyclopentene-1-hydroxymethyl acetate).

### Embodiment 18

The method of any one of embodiments 1 to 17, further comprising preparing CTOX by adding a haloacetic acid to a suspension of t-butanol and potassium tert-butoxide.

### Embodiment 19

The method of embodiment 18 wherein the haloactic acid is chloroacetic acid, bromoacetic acid, or iodoacteic acid.

### Embodiment 20

The method of embodiment 18 wherein the haloacetic acid is added to the suspension of t-butanol and potassium tert-butoxide slowly at about 30° C. to about 50° C.

### Embodiment 21

The method of embodiment 20 further comprising heating and maintaining the reaction mass of haloacetic acid, t-butanol, and potassium tert-butoxide at about 80° C. to about 85° C. for about 6 hours to about 8 hours.

### Embodiment 22

The method of embodiment 21 further comprising adding water to the reaction mass of haloacetic acid, t-butanol, and potassium tert-butoxide at about 30° C. to about 45° C., and concentrating the reaction mass under vacuum.

### Embodiment 23

The method of embodiment 22 further comprising adding water to the reaction mass at about 20° C. to about 35° C. and washing the reaction mass with methyl tert-butyl ether (MTBE).

### Embodiment 24

The method of embodiment 23 further comprising adjusting the aqueous layer pH to 1.5 to 3.0 with a sulfuric acid solution or a hydrochloric acid solution, and extracting the product with MTBE or diethyl ether.

### Embodiment 25

The method of embodiment 24 wherein the product is extracted with MTBE.

### Embodiment 26

The method of embodiment 24 or embodiment 25 further comprising washing the organic layer with an aqueous sodium chloride solution, and concentrating the organic layer under vacuum, to produce liquid CTOX.

In order that the subject matter disclosed herein may be more efficiently understood, examples are provided below. It should be understood that these examples are for illustrative purposes only and are not to be construed as limiting the claimed subject matter in any manner Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1: Preparation of ((1S, 4R)-4-(2-amino-6-(cyclopropylamino)-9H-purin-9-yl) cyclopent-2-en-1-yl) methyl 2-(tert-butoxy) acetate

To a suspension of Abacavir free base (75 g, 261.9 mmol), 2-(tert-butoxy) acetic acid (41.54 g, 314.3 mmol) and DMAP (48 g, 392.9 mmol) in DCM (825 mL) was added EDC-HCl (3*25.11 g, 393 mmol) lot wise and the mixture was stirred for 2-3 hours at 25-30° C. After reaction completion, added 10% ammonium chloride solution, separated the organic layer and concentrated under vacuum. Crude dissolved in toluene and washed with 15% aqueous acetic acid, followed by water, 5% aqueous sodium bicarbonate solution and finally 10% aqueous sodium chloride solution. The organic layer was concentrated under vacuum, dissolved in DCM (150 mL) and slowly added n-heptane (450 mL). The suspension was stirred for 10-12 hours at 25-30° C., followed by 5-6 hours at 0-5° C. The product was 5 filtered, washed with n-heptane (150 mL) and dried under vacuum to yield ((1S, 4R)-4-(2-amino-6-(cyclopropylamino)-9H-purin-9-yl) cyclopent-2-en-1-yl) methyl 2-(tert-butoxy) acetate as a white powder (yield 74%).

The purity by HPLC analysis was 99.3%. ¹H NMR (400 MHz, DMSO-d6): 7.60; (s, 1H), 7.27; (s, 1H), 6.08; (d, J=5.2 Hz, 1H), 5.96; (d, J=6.0 Hz, 1H), 5.81; (s, 2H), 5.42-5.41; (m, 1H), 4.16-4.13; (m, 2H), 3.99; (d, J=3.2 Hz, 2H), 3.11-3.08; (m, 1H), 3.11-3.08; (m, 1H), 2.69-2.65; (m, 1H,), 1.62-1.58; (m, 1H), 1.12; (s, 9H), 0.65-0.57; (m, 4H). IR (KBr): v 3327, 3191, 2973, 1764, 1648, 1610, 1487, 1265, and 1186 cm⁻¹.

### Example 2: Preparation of ((1S, 4R)-4-(2-amino-6-(cyclopropylamino)-9H-purin-9-yl) cyclopent-2-en-1-yl) methyl 2-hydroxyacetate/Prurisol™ ((−) cis-[4-[2-amino-6-(cyclopropylamino)-9H-purin-9-yl]-2-cyclopentene-1-hydroxymethyl acetate)

To the solution of ((1S, 4R)-4-(2-amino-6-(cyclopropylamino)-9H-purin-9-yl) cyclopent-2-en-1-yl) methyl 2-(tert-butoxy) acetate produced in Example 1 (50 g, 124.9 mmol) in DCM (400 mL) was added trifluoro acetic acid (250 g, 2192.5 mmol) at 0-5° C. and stirred at 25-30° C. for 4-6 hours. Reaction mass cooled to −10° C. and added triethyl amine (250 g, 2470.5 mmol) below 20° C. Reaction mass concentrated to around 50%, added water (500 mL), stirred at 20-30° C., filtered the product and washed with water. Wet material slurry washed with water followed by acetone and dried under vacuum to get Prurisol™ ((−) cis-[4-[2-amino-6-(cyclopropylamino)-9H-purin-9-yl]-2-cyclopentene-1-hydroxymethyl acetate) as a white powder (yield=73%, HPLC Purity=99.2%).

Crystallization: The above solid (20.0 g, 58.1 mmol) dissolved in mixture of acetone (252 mL) and water (102 mL) at 50-55° C. and cooled to 0-5° C. Product filtered, washed with acetone and dried under vacuum to get a Prurisol™ ((−) cis-[4-[2-amino-6-(cyclopropylamino)-9H-purin-9-yl]-2-cyclopentene-1-hydroxymethyl acetate) (CSRA) as a white powder (yield 80%). The purity by HPLC analysis was 99.68%. ¹H NMR (400 MHz, DMSO-d6): 7.60; (s, 1H), 7.28; (s, 1H), 6.08; (d, J=5.6 Hz, 1H), 5.95; (d, J=6.0 Hz, 1H), 5.81; (s, 2H), 5.41-5.38; (m, 1H), 5.33; (t, J=6.8 Hz, 1H), 4.15; (d, J=6.4 Hz, 2H), 4.01; (m, 2H), 3.09-3.03; (m, 1H), 3.09-3.03; (m, 1H), 2.71-2.63; (m, 1H), 1.62-1.56; (m, 1H), 0.65-0.57; (m, 4H). ¹³C NMR (100 MHz, DMSO-d6): 172.7, 160.0, 155.9, 151.0, 136.2, 134.7, 131.0, 113.6, 66.2, 59.5, 58.0, 43.9, 34.3, 23.7, 6.49. MS (ESI) m/z=345.16 [M H⁺]. IR (KBr): v 3339, 3229, 3171, 2950, 1732, 1638, 1617, 1489, 1212, 1105 cm⁻¹.

### Example 3: Side Chain: Preparation of 2-(tert-butoxy) acetic acid (CTOX):

To a suspension of t-butanol (1300 mL) and potassium tert-butoxide (262 g, 2335 mmol) was added chloroacetic acid (100 g, 1058.2 mmol) slowly at 30-50° C. Reaction mass was heated and maintained at 80-85° C. for 6-8 hours. Added water (200 mL) at 30-45° C. and concentrated under vacuum. Added water (800 mL) at 20-35° C. and washed with MTBE. Aqueous layer pH adjusted to 1.5-3.0 using 4 N sulfuric acid solution and extracted the product with MTBE. Organic layer washed with aqueous sodium chloride solution, concentrated the organic layer under vacuum, and obtained CTOX as liquid product (yield 62.9%; purity by GC=99.38%). ¹H NMR (400 MHz, CDCl3): 7.267; (s, 1H), 4.029 (s, 2H), 1.271; (s, 9H).

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A method of preparing a compound having the formula 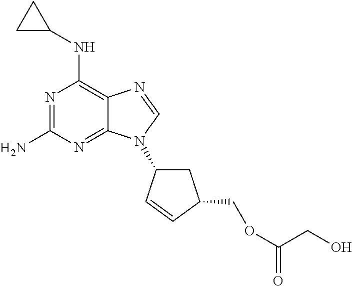 [SMILES: Nc1nc(NC2CC2)c2ncn([C@H]3C=C[C@@H](COC(=O)CO)C3)c2n1]
Prurisol™ ((−) cis-[4-[2-amino-6-(cyclopropylamino)-9H-purin-9-yl]-2-cyclopentene-1-hydroxymethyl acetate) comprising:
a) adding 1-ethyl-3-(3-dimethylaminopropyl)carbodiimide (EDC-HCl) to a suspension comprising Abacavir free base, a coupling agent, and 4-dimethylaminopyridine (DMAP) in a solvent under conditions sufficient to produce ((1S, 4R)-4-(2-amino-6-(cyclopropylamino)-9H-purin-9-yl)cyclopent-2-en-1-yl)methyl 2-(tert-butoxy) acetate; and
b) adding a strong acid to a solution of ((1S, 4R)-4-(2-amino-6-(cyclopropylamino)-9H-purin-9-yl) cyclopent-2-en-1-yl)methyl 2-(tert-butoxy) acetate in a solvent under conditions sufficient to produce Prurisol™ ((−) cis-[4-[2-amino-6-(cyclopropylamino)-9H-purin-9-yl]-2-cyclopentene-1-hydroxymethyl acetate).

2. The method of claim 1 wherein:
the coupling agent is 2-(tert-butoxy) acetic acid (CTOX), (benzotriazol-1-yloxy)tripyrrolidinophosphonium hexafluorophosphate (PyBOP), or [dimethylamino(triazolo[4,5-b]pyridin-3-yloxy)methylidene]-dimethylazanium hexafluorophosphate (HATU), or any combination thereof; or
the solvent is dichloromethane (DCM) or tetrahydrofuran (THF); or
the strong acid is trifluoro acetic acid (TFA).

3. The method of claim 1 further comprising:
c) dissolving Prurisol™ ((−) cis-[4-[2-amino-6-(cyclopropylamino)-9H-purin-9-yl]-2-cyclopentene-1-hydroxymethyl acetate) in a mixture of acetone and water under conditions sufficient to produce crystallized Prurisol™ ((−) cis-4-[2-amino-6-(cyclopropylamino)-9H-purin-9-yl]-2-cyclopentene-1-hydroxymethyl acetate).

4. The method of claim 1 further comprising stirring the reaction mixture of EDC-HCl, Abacavir free base, coupling agent, and DMAP in solvent at about 25° C. to about 30° C. for about 2 hours to about 3 hours.

5. The method of claim 4 further comprising adding a 10% ammonium chloride solution or a brine solution to the reaction mixture, separating the organic layer, and concentrating the organic layer under vacuum to produce a crude ((1S, 4R)-4-(2-amino-6-(cyclopropylamino)-9H-purin-9-yl)cyclopent-2-en-1-yl)methyl 2-(tert-butoxy) acetate reaction product.

6. The method of claim 5 further comprising dissolving the crude reaction product in toluene, washing the crude reaction product with 15% aqueous acetic acid, washing the crude reaction product with water, washing the crude reaction product with a 5% aqueous sodium bicarbonate solution, and washing the crude reaction product with a 10% aqueous sodium chloride solution.

7. The method of claim 6 further comprising concentrating the organic layer under vacuum, dissolving the organic layer in DCM or THF, slowly adding n-heptane or n-hexane, stirring the suspension at about 25° C. to about 30° C. for about 10 hours to about 12 hours, and stirring the suspension at about 0° C. to about 5° C. for about 5 to about 6 hours.

8. The method of claim 7 further comprising filtering the product, washing the product with n-heptane or n-hexane, and drying the product under vacuum to produce ((1S, 4R)-4-(2-amino-6-(cyclopropylamino)-9H-purin-9-yl)cyclopent-2-en-1-yl)methyl 2-(tert-butoxy) acetate.

9. The method of claim 1 wherein the strong acid is added to the solution of (1S, 4R)-4-(2-amino-6-(cyclopropylamino)-9H-purin-9-yl) cyclopent-2-en-1-yl)methyl 2-(tert-butoxy) acetate in DCM at about 0° C. to about 5° C. and is stirred at about 25° C. to about 30° C. for about 4 hours to about 6 hours.

10. The method of claim 9 further comprising cooling the reaction mass to −10° C., adding triethyl amine below 20° C., concentrating the reaction mass up to about 50%, adding water, and stirring at about 20° C. to about 30° C.

11. The method of claim 10 further comprising filtering the product and washing the product with water, followed by acetone and drying under vacuum to produce Prurisol™ ((−) cis-[4-[2-amino-6-(cyclopropylamino)-9H-purin-9-yl]-2-cyclopentene-1-hydroxymethyl acetate).

12. The method of claim 11 wherein the Prurisol™ ((−) cis-[4-[2-amino-6-(cyclopropylamino)-9H-purin-9-yl]-2-cyclopentene-1-hydroxymethyl acetate) solid is dissolved in mixture of acetone and water at about 50° C. to about 55° C. and is cooled to about 0° C. to about 5° C.

13. The method of claim 12 further comprising filtering the product, washing the product with acetone, and drying the product under vacuum to produce crystallized Prurisol™ ((−) cis-[4-[2-amino-6-(cyclopropylamino)-9H-purin-9-yl]-2-cyclopentene-1-hydroxymethyl acetate).

14. The method of claim 1 further comprising preparing 2-(tert-butoxy) acetic acid (CTOX) by adding a haloacetic acid to a suspension of t-butanol and potassium tert-butoxide.

15. The method of claim 14 wherein the haloacetic acid is added to the suspension of t-butanol and potassium tert-butoxide slowly at about 30° C. to about 50° C.

16. The method of claim 15 further comprising heating and maintaining the reaction mass of haloacetic acid, t-butanol, and potassium tert-butoxide at about 80° C. to about 85° C. for about 6 hours to about 8 hours.

17. The method of claim 16 further comprising adding water to the reaction mass of haloacetic acid, t-butanol, and potassium tert-butoxide at about 30° C. to about 45° C., and concentrating the reaction mass under vacuum.

18. The method of claim 17 further comprising adding water to the reaction mass at about 20° C. to about 35° C. and washing the reaction mass with methyl tert-butyl ether (MTBE).

19. The method of claim 18 further comprising adjusting the aqueous layer pH to 1.5 to 3.0 with a sulfuric acid solution or a hydrochloric acid solution, and extracting the product with MTBE or diethyl ether.

20. The method of claim 19 further comprising washing the organic layer with an aqueous sodium chloride solution, and concentrating the organic layer under vacuum, to produce liquid CTOX.
